SEQUENCE LISTING (3 sequences)

SEQ ID NO: 1 - DNA, 41 bp, Artificial Sequence
  Description of Artificial Sequence Synthetic primer
aacaacggaggaggaggaaaacagggcccctaggaaaaagg

SEQ ID NO: 2 - DNA, 39 bp, Artificial Sequence
  Description of Artificial Sequence Synthetic primer
gagcggataacaatttcacagtctcaatagggctaatgg

SEQ ID NO: 3 - DNA, 39 bp, Artificial Sequence
  Description of Artificial Sequence Synthetic oligonucleotide
tgaaattgttatccgctcaacaacggaggaggaggaaaa

(19) United States
(12) Patent Application Publication
(10) Pub. No.: US 2020/0308576 A1
(43) Pub. Date: Oct. 1, 2020

(54) NOVEL METHOD FOR GENERATING CIRCULAR SINGLE-STRANDED DNA LIBRARIES

(71) Applicants: Roche Sequencing Solutions, Inc., Pleasanton, CA (US); Kapa Biosystems, Inc., Wilmington, MA (US)

(72) Inventors: Daleen Badenhorst, Wellington (ZA); Richard Dannebaum, Pleasant Hill, CA (US); Ashley Hayes, San Francisco, CA (US); Monica Herrera, Livermore, CA (US); Severine Margeridon, Castro Valley, CA (US); Martin Ranik, Cape Town (ZA)

(21) Appl. No.: 16/808,173

(22) Filed: Mar. 3, 2020

Related U.S. Application Data
(63) Continuation of application No. PCT/EP2018/074761, filed on Sep. 13, 2018.
(60) Provisional application No. 62/558,753, filed on Sep. 14, 2017.

(51) Int. Cl. C12N 15/10; C12Q 1/6874

(52) U.S. Cl. CPC C12N 15/1068; C12Q 1/6874

(57) ABSTRACT

The invention is a novel method of making and using a library such as a sequencing library of single stranded circular nucleic acid templates via splint ligation.

### CROSS-REFERENCE TO RELATED APPLICATIONS

This application is a continuation of the International Application Serial No. PCT/EP/2018/074761 filed on Sep. 13, 2018, which claims priority to the U.S. Provisional Application Ser. No. 62/558,753 filed on Sep. 14, 2017 both of which are incorporated herein by reference.

### FIELD OF THE INVENTION

The invention relates to the field of nucleic acid analysis and more specifically, to preparing circular templates for nucleic acid sequencing.

### BACKGROUND OF THE INVENTION

Circular nucleic acid templates have multiple uses in nucleic acid analysis. Linear nucleic acids are converted into a circular form for amplification, e.g., by rolling circle amplification (RCA) and subsequent detection and quantification, see U.S. Pat. No. RE44265. The use of circular templates in sequencing is also known in the art. See U.S. Pat. Nos. 7,302,146 and 8,153,375. Current sequencing strategies also require that auxiliary sequences such as primer binding sites and barcodes be introduced into a template. The present invention is a novel efficient method of creating circular nucleic acid templates suitable for sequencing. The method allows the creation of templates of virtually unrestricted length.

### SUMMARY OF THE INVENTION

In some embodiments, the invention is a method of forming a circular molecule from a target nucleic acid, comprising: amplifying the target nucleic acid with a first and second bipartite amplification primers comprising a universal circularization sequence and a target-specific sequence to generate double stranded amplicons; separating the strands of the double stranded amplicons; contacting the strand of the amplicon with a circularization oligonucleotide to generate a hybrid structure wherein the universal circularization sequences in the strand are hybridized to the circularization oligonucleotide so that the ends of the strand are brought into ligatable proximity; and ligating the ends of the strand thereby forming a circular molecule. The target nucleic acid may comprise fragments of a genome selected from cell-free plasma DNA, sonicated DNA and restriction digested DNA. In some embodiments, the universal sequences on the first and second amplification primers are distinct. The universal sequence of the first or the second amplification primer may comprise a sequencing primer. In some embodiments, the universal primers comprise SED ID Nos: 1 and 2. In some embodiments only one of the first and second amplification primers comprises a 5′-phosphate group.

In some embodiments, the strands of the double stranded amplicons are separated by nuclease digestion or by physical means.

In some embodiments, the circularization oligonucleotide comprises a ligand for a capture moiety. In some embodiments, the ligand-capture moiety pair is selected from biotin-streptavidin, antibody-antigen or oligonucleotide-complementary capture oligonucleotide. In some embodiments, the circularization oligonucleotide comprises SED ID NO: 3. In some embodiments the circularization oligonucleotide is a Y-shaped structure with single-stranded regions complementary to the universal circularization sequences in the bipartite primers.

In some embodiments, the invention is making a library of circular target nucleic acids for sequencing comprising: amplifying the target nucleic acids with a first and second bipartite amplification primers comprising a universal circularization sequence and a target-specific sequence to generate double stranded amplicons; separating the strands of the double stranded amplicons; contacting the strands with a circularization oligonucleotide to generate hybrid structures wherein the universal circularization sequences in the strands are hybridized to the circularization oligonucleotides so that the ends of each strand are brought into ligatable proximity; and ligating the ends of the strands thereby forming a library of circular target nucleic acids. In some embodiments, the target nucleic acids comprise a universal adaptor sequence comprising universal primer binding sites conjugated to the target sequence.

In some embodiments, the invention is a method of determining the sequence of a double-stranded target nucleic acid in a sample comprising: attaching universal primer binding sites to the ends of the target nucleic acid in a sample to form adapted target nucleic acid; amplifying the adapted target nucleic acid with a first and second bipartite amplification primers comprising a universal primer complementary to the universal primer binding site and a universal circularization sequence to generate double stranded amplicons; separating the strands of the double stranded amplicons; contacting the strands with a circularization oligonucleotide to generate hybrid structures wherein the universal circularization sequences in the strands are hybridized to the circularization oligonucleotides so that the ends of each strand are brought into ligatable proximity; ligating the ends of the strands thereby forming a circular target nucleic acid; contacting the sample with a sequencing primer complementary to one of the universal sequences of the bipartite primers; and extending the sequencing primer with a nucleic acid polymerase thereby determining the sequence of the target nucleic acid. In some embodiments, the universal priming sites are attached via ligation of an adaptor comprising the universal priming sites.

In some embodiments, the invention is a method of determining the sequence of a double-stranded target nucleic acid in a sample comprising: amplifying the target nucleic acid with a first and second bipartite amplification primers comprising a target-specific sequence and a universal circularization sequence to generate double stranded amplicons; separating the strands of the double stranded amplicons; contacting the strands with a circularization oligonucleotide to generate hybrid structures wherein the universal circularization sequences in the strands are hybridized to the circularization oligonucleotides so that the ends of each strand are brought into ligatable proximity; and; ligating the ends of the strands thereby forming a circular target nucleic acid; contacting the sample with a sequencing primer complementary to one of the universal sequences of the bipartite primers; and extending the sequencing primer with a nucleic acid polymerase thereby determining the sequence of the target nucleic acid.

In some embodiments, the invention is a kit for determining the sequence of a target nucleic acid comprising: a first and second bipartite amplification primers comprising a universal circularization sequence and a target-binding sequence; a circularization oligonucleotide at least partially complementary to the universal circularization sequences in the bipartite primers so that the ends of the strands comprising the bipartite primers can be brought ligatable proximity. In some embodiments, the kit also comprises a DNA polymerase and DNA ligase. In some embodiments, only one of the first and second bipartite amplification primers is phosphorylated at the 5′-end. In some embodiments, the circularization oligonucleotide comprises a ligand for a capture moiety. In some embodiments, the circularization oligonucleotide is a Y-shaped structure with single-stranded regions complementary to the universal circularization sequences in the bipartite primers.

### BRIEF DESCRIPTION OF THE DRAWINGS

FIG. 1 shows the general scheme of the circularization method.

FIG. 2 shows a detailed scheme of the circularization method.

FIG. 3 shows yield of the single stranded circular DNA under various conditions.

FIG. 4 shows results of sequencing of the single-stranded DNA libraries.

### DETAILED DESCRIPTION OF THE INVENTION

### Definitions

The following definitions aid in understanding of this disclosure.

The term “sample” refers to any composition containing or presumed to contain target nucleic acid. This includes a sample of tissue or fluid isolated from an individual for example, skin, plasma, serum, spinal fluid, lymph fluid, synovial fluid, urine, tears, blood cells, organs and tumors, and also to samples of in vitro cultures established from cells taken from an individual, including the formalin-fixed paraffin embedded tissues (FFPET) and nucleic acids isolated therefrom. A sample may also include cell-free material, such as cell-free blood fraction that contains cell-free DNA (cfDNA) or circulating tumor DNA (ctDNA).

The term “nucleic acid” refers to polymers of nucleotides (e.g., ribonucleotides and deoxyribonucleotides, both natural and non-natural) including DNA, RNA, and their subcategories, such as cDNA, mRNA, etc. A nucleic acid may be single-stranded or double-stranded and will generally contain 5′-3′ phosphodiester bonds, although in some cases, nucleotide analogs may have other linkages. Nucleic acids may include naturally occurring bases (adenosine, guanosine, cytosine, uracil and thymidine) as well as non-natural bases. Some examples of non-natural bases include those described in, e.g., Seela et al., (1999) Helv. Chim. Acta 82:1640. The non-natural bases may have a particular function, e.g., increasing the stability of the nucleic acid duplex, inhibiting nuclease digestion or blocking primer extension or strand polymerization.

The terms “polynucleotide” and “oligonucleotide” are used interchangeably. Polynucleotide is a single-stranded or a double-stranded nucleic acid. Oligonucleotide is a term sometimes used to describe a shorter polynucleotide. An oligonucleotide may be comprised of at least 6 nucleotides or about 15-50 nucleotides. Oligonucleotides are prepared by any suitable method known in the art, for example, by a method involving direct chemical synthesis as described in Narang et al. (1979) Meth. Enzymol. 68:90-99; Brown et al. (1979) Meth. Enzymol. 68:109-151; Beaucage et al. (1981) Tetrahedron Lett. 22:1859-1862; Matteucci et al. (1981) J. Am. Chem. Soc. 103:3185-3191.

The term “primer” refers to a single-stranded oligonucleotide which hybridizes with a sequence in the target nucleic acid (“primer binding site”) and is capable of acting as a point of initiation of synthesis along a complementary strand of nucleic acid under conditions suitable for such synthesis.

The term “adaptor” means a nucleotide sequence that may be added to another sequence so as to import additional properties to that sequence. An adaptor is typically an oligonucleotide that can be single- or double-stranded, or may have both a single-stranded portion and a double-stranded portion.

The term “ligation” refers to a condensation reaction joining two nucleic acid strands wherein a 5′-phosphate group of one molecule reacts with the 3′-hydroxyl group of another molecule. Ligation is typically an enzymatic reaction catalyzed by a ligase or a topoisomerase. Ligation may join two single strands to create one single-stranded molecule. Ligation may also join two strands each belonging to a double-stranded molecule thus joining two double-stranded molecules. Ligation may also join both strands of a double-stranded molecule to both strands of another double-stranded molecule thus joining two double-stranded molecules. Ligation may also join two ends of a strand within a double-stranded molecule thus repairing a nick in the double-stranded molecule.

The term “barcode” refers to a nucleic acid sequence that can be detected and identified. Barcodes can be incorporated into various nucleic acids. Barcodes are sufficiently long e.g., 2, 5, 20 nucleotides, so that in a sample, the nucleic acids incorporating the barcodes can be distinguished or grouped according to the barcodes.

The term “multiplex identifier” or “MID” refers to a barcode that identifies a source of a target nucleic acids (e.g., a sample from which the nucleic acid is derived). All or substantially all the target nucleic acids from the same sample will share the same MID. Target nucleic acids from different sources or samples can be mixed and sequenced simultaneously. Using the MIDs the sequence reads can be assigned to individual samples from which the target nucleic acids originated.

The term “unique molecular identifier” or “UID” refers to a barcode that identifies a nucleic acid to which it is attached. All or substantially all the target nucleic acids from the same sample will have different UIDs. All or substantially all of the progeny (e.g., amplicons) derived from the same original target nucleic acid will share the same UID.

The term “universal primer” and “universal priming binding site” or “universal priming site” refer to a primer and primer binding site present in (typically, through in vitro addition to) different target nucleic acids. The universal priming site is added to the plurality of target nucleic acids using adaptors or using target-specific (non-universal) primers having the universal priming site in the 5′-portion. The universal primer can bind to and direct primer extension from the universal priming site.

More generally, the term “universal” refers to a nucleic acid molecule (e.g., primer or other oligonucleotide) that can be added to any target nucleic acid and perform its function irrespectively of the target nucleic acid sequence. The universal molecule may perform its function by hybridizing to the complement, e.g., a universal primer to a universal primer binding site or a universal circularization oligonucleotide to a universal primer sequence.

As used herein, the terms “target sequence”, “target nucleic acid” or “target” refer to a portion of the nucleic acid sequence in the sample which is to be detected or analyzed. The term target includes all variants of the target sequence, e.g., one or more mutant variants and the wild type variant.

The term “sequencing” refers to any method of determining the sequence of nucleotides in the target nucleic acid.

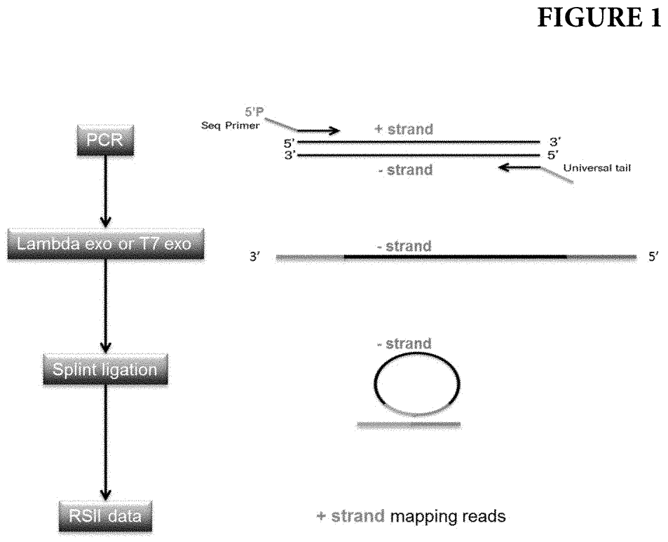

The present invention is a method of making circular target nucleic acid molecules and libraries of such molecules for downstream analysis such as nucleic acid sequencing. As shown in FIG. 1, the method comprises the use an oligonucleotide probe to circularize nucleic acid molecules. First, the nucleic acid molecules have universal sequences added to each end. Nucleic acids with universal sequences at each end are then rendered single stranded and contacted with a probe complementary to at least a portion of the universal sequences. The probe is hybridized to enable circularization and formation of single stranded circular (sscDNA) molecules.

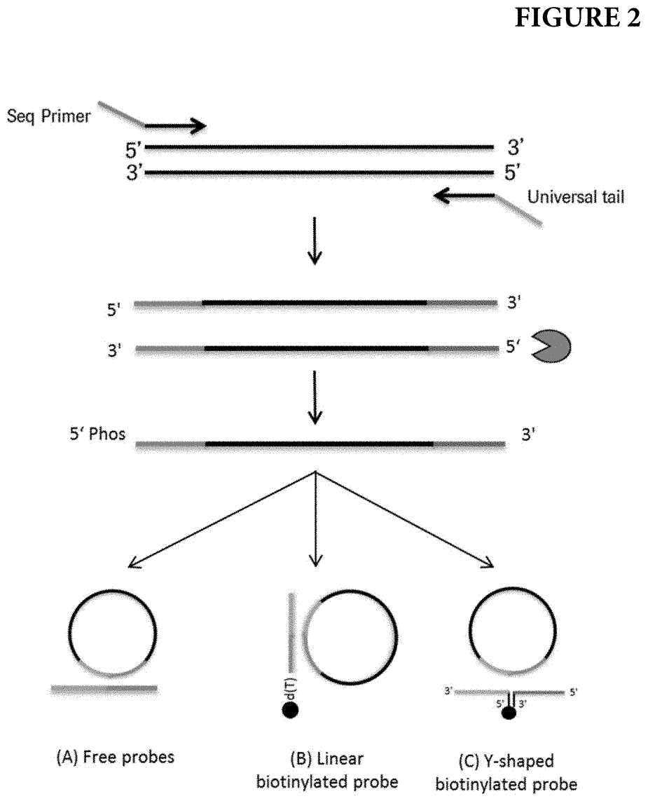

The method has advantages over existing circularization methods, e.g., U.S. RE44265 and US2012003657. In contrast to that method, the present method uses a universal circularization sequence attached to the target sequences. The present method does not use a non-target oligonucleotide containing multiple restriction sites inserted between the ends of the target molecule to ensure the presence of restriction sites (see U.S. RE44265, FIG. 2 therein). The same strategy is used in US2012003657 (see FIG. 1A therein) where the “vector” oligonucleotide containing sequencing primer binding sites is used. The present invention uses more efficient intramolecular circularization instead of intermolecular ligation with auxiliary oligonucleotides.

The present invention comprises detecting a target nucleic acid in a sample. In some embodiments, the sample is derived from a subject or a patient. In some embodiments the sample may comprise a fragment of a solid tissue or a solid tumor derived from the subject or the patient, e.g., by biopsy. The sample may also comprise body fluids (e.g., urine, sputum, serum, plasma or lymph, saliva, sputum, sweat, tear, cerebrospinal fluid, amniotic fluid, synovial fluid, pericardial fluid, peritoneal fluid, pleural fluid, cystic fluid, bile, gastric fluid, intestinal fluid, and/or fecal samples), The sample may comprise whole blood or blood fractions where tumor cells may be present. In some embodiments, the sample, especially a liquid sample may comprise cell-free material such as cell-free DNA or RNA including cell-free tumor DNA or tumor RNA. In some embodiments, the sample is a cell-free sample, e.g., cell-free blood-derived sample where cell-free tumor DNA or tumor RNA are present. In other embodiments, the sample is a cultured sample, e.g., a culture or culture supernatant containing or suspected to contain an infectious agent or nucleic acids derived from the infectious agent. In some embodiments, the infectious agent is a bacterium, a protozoan, a virus or a mycoplasma.

A target nucleic acid is the nucleic acid of interest that may be present in the sample. In some embodiments, the target nucleic acid is a gene or a gene fragment. In other embodiments, the target nucleic acid contains a genetic variant, e.g., a polymorphism, including a single nucleotide polymorphism or variant (SNP of SNV), or a genetic rearrangement resulting e.g., in a gene fusion. In some embodiments, the target nucleic acid comprises a biomarker. In other embodiments, the target nucleic acid is characteristic of a particular organism, e.g., aids in identification of the pathogenic organism or a characteristic of the pathogenic organism, e.g., drug sensitivity or drug resistance. In yet other embodiments, the target nucleic acid is characteristic of a human subject, e.g., the HLA or KIR sequence defining the subject's unique HLA or KIR genotype. In yet other embodiments, all the sequences in the sample are target nucleic acids e.g., in shotgun genomic sequencing.

In an embodiment of the invention, a double-stranded target nucleic acid is converted into the template configuration of the invention. In some embodiments, the target nucleic acid occurs in nature in a single-stranded form (e.g., RNA, including mRNA, microRNA, viral RNA; or single-stranded viral DNA). The single-stranded target nucleic acid is converted into double-stranded form to enable the further steps of the claimed method.

Longer target nucleic acids may be fragmented although in some applications longer target nucleic acids may be desired to achieve a longer read. In some embodiments, the target nucleic acid is naturally fragmented, e.g., circulating cell-free DNA (cfDNA) or chemically degraded DNA such as the one founds in preserved samples. In other embodiments, the target nucleic acid is fragmented in vitro, e.g., by physical means such as sonication or by endonuclease digestion, e.g., restriction digestion.

In some embodiments, the invention is a method comprising a step of amplifying the target nucleic acid. The amplification may be by polymerase chain reaction (PCR) or any other method that utilizes oligonucleotide primers. Various PCR conditions are described in PCR Strategies (M. A. Innis, D. H. Gelfand, and J. J. Sninsky eds., 1995, Academic Press, San Diego, CA) at Chapter 14; PCR Protocols: A Guide to Methods and Applications (M. A. Innis, D. H. Gelfand, J. J. Sninsky, and T. J. White eds., Academic Press, NY, 1990).

The amplification may utilize first and second bipartite amplification primers comprising a universal circularization sequence and a target-specific sequence to generate double stranded amplicons. (FIG. 2). In some embodiments, a defined target or group of target nucleic acids is being interrogated. In such embodiments, target specific amplification primers may be used. A primer may have a bipartite structure composed of a target-specific sequence in the 3′-portion and a universal sequence in the 5′-portion of the primer. Typically, the target-specific primers are used as a pair of distinct oligonucleotides, e.g., a forward and a reverse primer. For subsequent steps, a different universal sequence can be added to the 5′-portion of the forward and the reverse primer in order to distinguish the complementary strands (i.e., the (+) and the (−) strands) in subsequent steps of the method. In some embodiments, the universal sequence of the bipartite primers comprises a sequencing primer binding site.

The amplification may also utilize a universal adaptor sequence comprising universal primer binding sites conjugated to the target sequence In other embodiments, a plurality of target nucleic acids is being interrogated, e.g.., a whole genome or all nucleic acids present in a sample, e.g., a sample suspected of containing one or more unknown pathogenic organisms. In such embodiments, a target specific primer is not advantageous and a universal primer is used. In such embodiments, a universal primer binding site is added, e.g., by ligation of an adaptor molecule containing a universal primer binding site sequence. Typically, such adaptors are added independent of the sequence of the target nucleic acid, for example, by ligation. In such embodiments, the target nucleic acids receive the same adaptor molecule at each end. To distinguish the strands of the resulting adapted target nucleic acid, the adaptor may have a Y-structure, see e.g., U.S. Pat. Nos. 8,053,192, 8,182,989 and 8,822,150.

In some embodiments of the present invention, the adaptor molecules are ligated to the target nucleic acid. The ligation can be a blunt-end ligation or a more efficient cohesive-end ligation. The target nucleic acid or the adaptors may be rendered blunt-ended by strand-filling, i.e., extending a 3′-terminus by a DNA polymerase to eliminate a 5′-overhang. In some embodiments, the blunt-ended adaptors and target nucleic acid may be rendered cohesive by addition of a single nucleotide to the 3′-end of the adaptor and a single complementary nucleotide to the 3′-ends of the target nucleic acid, e.g., by a DNA polymerase or a terminal transferase. In yet other embodiments, the adaptors and the target nucleic acid may acquire cohesive ends (overhangs) by digestion with restriction endonucleases. The latter option is more advantageous for known target sequences that are known to contain the restriction enzyme recognition site. In each of the above embodiments, the adaptor molecule may acquire the desired ends (blunt, single-base extension or multi-base overhang) by design of the synthetic adaptor oligonucleotides further described below. In some embodiments, other enzymatic steps may be required to accomplish the ligation. In some embodiments, a polynucleotide kinase may be used to add 5′-phosphates to the target nucleic acid molecules and adaptor molecules.

In some embodiments, the adaptor molecules are in vitro synthesized artificial sequences. In other embodiments, the adaptor molecules are in vitro synthesized naturally-occurring sequences known to possess the desired secondary structure. In yet other embodiments, the adaptor molecules are isolated naturally occurring molecules or isolated non naturally-occurring molecules.

In some embodiments, the invention comprises introduction of barcodes into the target nucleic acids. Sequencing individual molecules typically requires molecular barcodes such as described e.g., in U.S. Pat. Nos. 7,393,665, 8,168,385, 8,481,292, 8,685,678, and 8,722,368. A unique molecular barcode is a short artificial sequence added to each molecule in a sample such as a patient's sample typically during the earliest steps of in vitro manipulations. The barcode marks the molecule and its progeny. The unique molecular barcode (UID) has multiple uses. Barcodes allow tracking each individual nucleic acid molecule in the sample to assess, e.g., the presence and amount of circulating tumor DNA (ctDNA) molecules in a patient's blood in order to detect and monitor cancer without a biopsy. Unique molecular barcodes can also be used for sequencing error correction. The entire progeny of a single target molecule is marked with the same barcode and forms a barcoded family. A variation in the sequence not shared by all members of the barcoded family is discarded as an artifact and not a true mutation. Barcodes can also be used for positional deduplication and target quantification, as the entire family represents a single molecule in the original sample. See U.S. patent application Ser. Nos. 14/209,807 and 14/774,518.

In some embodiments of the present invention, bi-partite amplification primers comprise one or more barcodes. In other embodiments, adaptors comprise one or more barcodes. A barcode can be a multiplex sample ID (MID) used to identify the source of the sample where samples are mixed (multiplexed). The barcode may also serve as a unique molecular ID (UID) used to identify each original molecule and its progeny. The barcode may also be a combination of a UID and an MID. In some embodiments, a single barcode is used as both UID and MID.

In some embodiments, each barcode comprises a predefined sequence. In other embodiments, the barcode comprises a random sequence. Barcodes can be 1-20 nucleotides long.

In some embodiments, the method interrogates only one of the two strands of the target nucleic acid. The invention comprises a step of separating the strands of the double stranded amplicons. In some embodiments, one strand is degraded and the other strand is retained for subsequent steps of the method. In some embodiments, the amplicon is subjected to exonuclease treatment (e.g., by a viral exonuclease, T7 or Lambda exonuclease). In some embodiments, the primers or adapters may be modified to include a 5′- or 3′-end protection (such as a phosphorothioate) to specifically target the alternate strand for exonuclease digestion. The two strands may also be separated by physical means, i.e., alkaline denaturation or heat denaturation. In yet other embodiments, a desired strand is captured with an affinity reagent capable of selectively binding a strand with the affinity ligand. In some embodiments, a primer is biotinylated and the strand is captures with streptavidin.

In some embodiments, the ends of the target nucleic acid are phosphorylated. In some embodiments, the 5′-end of one primer is phosphorylated in order to effect degradation of one strand with an exonuclease, e.g., Lambda exonuclease. In other embodiments, the 5′-end of the adaptor is phosphorylated for that purpose. A mixture (e.g., an equal mixture) of phosphorylated and non-phosphorylated adaptors can be used to ensure that a single 5′-end of the adapted target molecule is phosphorylated.

In other embodiments, phosphorylation is necessary for the subsequent ligation step. Phosphorylation of the primer, the adaptor or the single-stranded molecule following the strand separation step can be performed e.g., with the use of a polynucleotide kinase (PNK) such as T4 PNK.

In some embodiments, the method includes a circularization step. This step includes contacting the 5′-phorphorylated strand of the amplicon with a circularization oligonucleotide to generate a hybrid structure wherein the universal circularization sequences in the strand are hybridized to the circularization oligonucleotide so that the ends of the strand are brought into ligatable proximity. The circularization oligonucleotide can be a linear oligonucleotide or a Y-shaped combination of two oligonucleotides. The Y-shaped structure comprises single-stranded regions complementary to the universal circularization sequences in the bipartite primers. The circularization oligonucleotide may also comprise a capture moiety for capture by an affinity reagent in a capture pair, such as a biotin-streptavidin, antibody-antigen or capture oligonucleotide-complementary oligonucleotide. The circularization oligonucleotide may be free in solution or bound to a solid support e.g., by a capture moiety described above. The capture may also occur after hybridization or after the ligation step described below.

In some embodiments, the invention further comprises a ligation step comprising ligating the ends of the strand hybridized to the circularization oligonucleotide thereby forming a circular molecule. The 5′-end of the strand is phosphorylated enabling the ligation step.

In some embodiments, the invention comprises an exonuclease digestion step wherein the linear nucleic acids possibly comprising excess oligonucleotides or un-circularized amplicons are removed from the reaction mixture. The final product is a circular ssDNA template containing the sequencing primer binding site.

In some embodiments, the invention is a method of making a library of circular target nucleic acids. The method comprises an amplification step with universal primers. The universal primer binding sites are added to the nucleic acids in the sample, e.g., by adaptor ligation to create a library of adapted molecules. The molecules in the library comprise target sequences flanked by universal sequence, e.g., universal primer binding site and a sequencing primer binding site. The circularization oligonucleotide may be complementary to the sequences contained in the adaptors. In other embodiments, the adaptors comprise only universal primer binding sites and universal primers introduce additional sequences not present in the adaptors. The universal primers may be bipartite amplification primers comprising a universal primer binding site and e.g., a sequencing primer binding site. The amplicons are then subjected to the steps of the method described above to generate a library of single stranded molecules.

In some embodiments, the present invention comprises detecting target nucleic acids in a sample by nucleic acid sequencing. Multiple nucleic acids, including all the nucleic acids in a sample may be converted into the template configuration of the invention and sequenced. In some embodiments, the library of circular molecules described herein can be subjected to nucleic acid sequencing.

Sequencing can be performed by any method known in the art. Especially advantageous is the high-throughput single molecule sequencing. Examples of such technologies include the Illumina HiSeq platform (Illumina, San Diego, Calif.), Ion Torrent platform (Life Technologies, Grand Island, N.Y.), Pacific BioSciences platform utilizing the SMRT (Pacific Biosciences, Menlo Park, Calif.) or a platform utilizing nanopore technology such as those manufactured by Oxford Nanopore Technologies (Oxford, UK) or Roche Sequencing Solutions (Santa Clara, Cal.) and any other presently existing or future DNA sequencing technology that does or does not involve sequencing by synthesis. The sequencing step may utilize platform-specific sequencing primers. Binding sites for these primers may be introduced in the method of the invention as described herein, i.e., by being a part of adaptors or amplification primers. In some embodiments, the sequencing platform does not require a specific sequencing primer and sequencing primer binding site is not introduced into the circular molecule.

In some embodiments, the invention is a method of determining the sequence of a double-stranded target nucleic acid. In this embodiment, the ligated single stranded circular nucleic acid is contacted with a sequencing primer complementary to the sequencing primer binding site present in the ssDNA and extending the sequencing primer with a nucleic acid polymerase thereby determining the sequence of the target nucleic acid.

The method of the invention enables the inclusion of a sequencing primer binding site in the final product (the single stranded circular target nucleic acid molecule) which allows for direct sequencing of the target molecule. With such a construction, it is possible to sequence the same strand of ssDNA multiple times and thus generate a consensus sequence. Notably, the method of the invention is applicable to a wide variety of target nucleic acid sizes. In some embodiments, the target nucleic acid is as short as 100 base pairs and as long as 10 kilobases.

In some embodiments, the sequencing step involves sequence analysis including a step of sequence aligning. In some embodiments, aligning is used to determine a consensus sequence from a plurality of sequences, e.g., a plurality having the same barcodes (UID). In some embodiments barcodes (UIDs) are used to determine a consensus from a plurality of sequences all having an identical barcode (UID). In other embodiments, barcodes (UIDs) are used to eliminate artifacts, i.e., variations existing in some but not all sequences having an identical barcode (UID). Such artifacts resulting from PCR errors or sequencing errors can be eliminated.

In some embodiments, the number of each sequence in the sample can be quantified by quantifying relative numbers of sequences with each barcode (UID) in the sample. Each UID represents a single molecule in the original sample and counting different UIDs associated with each sequence variant can determine the fraction of each sequence in the original sample. A person skilled in the art will be able to determine the number of sequence reads necessary to determine a consensus sequence. In some embodiments, the relevant number is reads per UID (“sequence depth”) necessary for an accurate quantitative result. In some embodiments, the desired depth is 5-50 reads per UID.

In some embodiments, the invention is a kit for performing the method of the invention. The kit comprises a first and second bipartite amplification primers comprising a target-binding sequence and optionally, universal sequence complementary to the circularization oligonucleotide; a circularization oligonucleotide at least partially complementary to both universal circularization sequences in the bipartite primers so that the ends of the strands comprising the bipartite primers can be brought ligatable proximity. The kit may also comprise a DNA ligase (in some embodiments, T4 DNA ligase, Taq DNA ligase, or E. coli DNA ligase is used), a polynucleotide kinase and a DNA polymerase, such as an amplification polymerase or a sequencing polymerase. Non-limiting examples of polymerases include prokaryotic DNA polymerases (e.g. Pol I, Pol II, Pol III, Pol IV and Pol V), eukaryotic DNA polymerase, archaeal DNA polymerase, telomerase, reverse transcriptase and RNA polymerase. Reverse transcriptase is an RNA-dependent DNA polymerase which synthesizes DNA from an RNA template. The reverse transcriptase family contains both DNA polymerase functionality and RNase H functionality, which degrades RNA base-paired to DNA.

In some embodiments, the DNA polymerase possesses strand displacement activity and does not have a 5′-3-exonuclease activity. In some embodiments, Phi29 polymerase and its derivatives are used, see U.S. Pat. Nos. 5,001,050, 5,576,204, 7,858,747 and 8,921,086. In some embodiments, the polymerase has the 3′-5′ exonuclease activity that advantageously removes the 3′-A overhang from the amplicon strands. In some embodiments, a recombinant Pyrococcus-derived high fidelity DNA polymerase with 5′-3′ and 3′-5′ exonuclease activity capable of generating blunt-ended products. See Frey, B. and Suppman, B. (1995). BioChemica. 2, 34-35.

### EXAMPLES

### Example 1

### Forming Single Stranded Circles from HIV-B Reference Sequence

DNA material used for testing is synthetic plasmid DNA ordered from Genewiz. The plasmid was designed from an HIV-B reference sequence to target the pol gene region of about 3.2 kb with a vector backbone pUC57 for cloning methods (in total ˜6.2 kb). The plasmid was transformed into E. coli competent cells, cloned, extracted and purified using standard procedures. Purified plasmid DNA was linearized with restriction enzyme and digested plasmid DNA was quantified using Bioanalzyer and diluted to 10⁸ copies/mL for PCR amplification.

The following primers were used:

SEQ ID NO: 1
/P/AACAACGGAGGAGGAGGAAAACAGGGCCCCTAGGAAAAAGG
SEQ ID NO: 2
GAGCGGATAACAATTTCACAGTCTCAATAGGGCTAATGG

Each 50 uL PCR reaction comprised Phusion DNA polymerase (New England BioLabs, Ipswich, Mass), forward and reserve taerget specific primers and 10⁶ copies of HIV Genewiz DNA template.

PCR amplification was performed in a thermocyder per manufacturer's recommendations. PCR QC Assessment using Fragment Analyzer or Bioanalyzer and Qubit dsDNA Broad Range to determine if there is any off-target product and the PCR reaction was efficient. The PCR products were purified to remove excess primers and PCR reagents using SPRI beads. Each bead cleanup was eluted in TrisHCl pH 8.0 and the volume amount for input of 2 ug into the exonuclease reaction was calculated.

The exonudease reaction with Lambda exonudease comprised Lamba exonudease and 2 ug of the dsDNA amplicon. The reaction was incubated at 37° C. for 30 min in a thermocycler with the heated lid followed by heat inactivation at 75° C. for 10 min.

The products were purified with SPRI beads in preparation for the next reaction in the workflow, eluted in 30 uL of elution buffer and measured on Qubit ssDNA and dsDNA kits as well as Bioanalyzer to determine the efficiency of the exonuclease reaction.

Phosphorylation was performed with T4 polynucleotide kinase. Phosphorylation of the single stranded DNA template will enable ligation.

The products were purified with SPRI beads in preparation for the next reaction in the workflow and eluted in 30 uL of elution buffer and proceed directly into the - ligation (circularization) reaction without the need for QC step.

Ligation was performed with DNA ligase and circularization oligonucleotide. A long probe with complementary sequences to the Primer sequence tail and the M13 tail is used for the circularization.

SEQ ID NO: 3
TGAAATTGTTATCCGCTCAACAACGGAGGAGGAGGAAAA

The 5′-Phosphorylated ssDNA template is mixed with the 10 ul of 20 uM linear probe in 49 uL volume and Taq DNA ligase is added in order to allow the circularization and ligation to occur.

The post-ligation products were purified with SPRI beads in preparation for the next reaction in the workflow and eluted in 45 uL of elution buffer. The post-ligation product is measured on Qubit ssDNA and dsDNA kit.

In the final step of preparing single stranded circular target nucleic acids, the linear or non-circularized nucleic acids were removed with a mixture of exonucleases Exo I and Exo III or with Exo VII. The reaction was incubated at 37 C for 30 minutes.

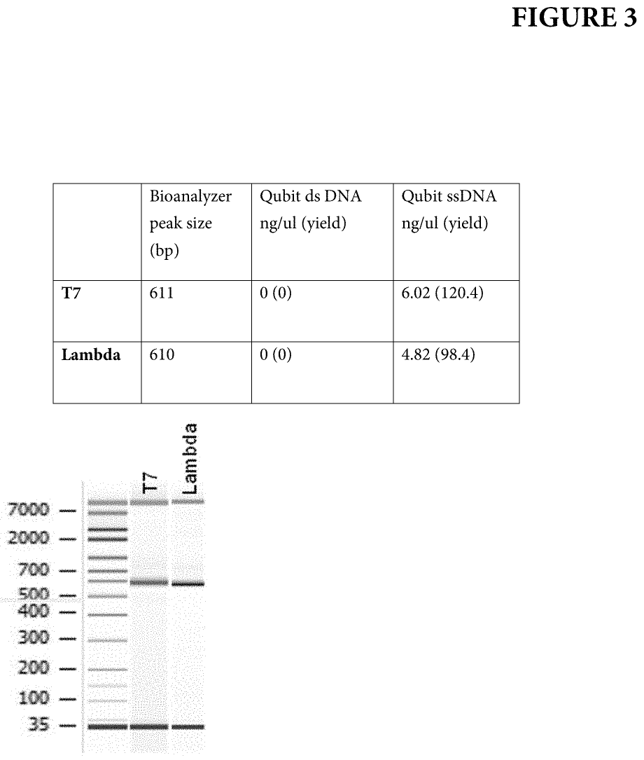

The products were purified with SPRI beads, eluted in 20 uL elution buffer and QC with Qubit ssDNA and dsDNA as well as Bioanalzyer High Sensitivity to determine yields and sizes of purified final libraries. Exemplary ss and dsDNA library yields are shown on FIG. 3.

### Example 2

### Sequencing Single-Stranded Circular Templates

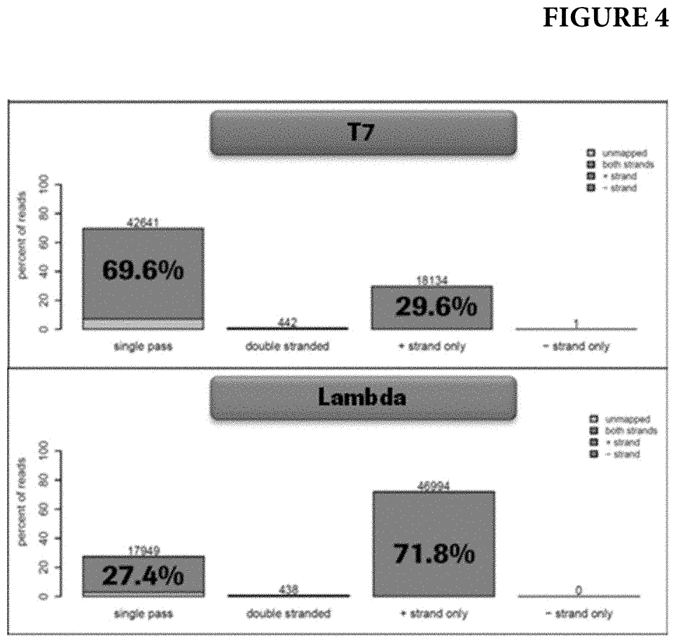

The single stranded circular molecules described above were sequenced on a Pacific BioSciences RSII instrument (Pacific BioSciences, Menlo Park, Calif.) according to the manufacturer's instructions. The results are shown in Table 1 and FIG. 4.

TABLE 1

| | Polymerase | | | |
|---|---|---|---|---|
| Library ID | read length | Empty (P0) | Productive (P1) | Other (P2) |
| T7 | 7373 | 57722 (38%) | 59226 (39%) | 33344 (22%) |
| Lambda | 15081 | 68401 (46%) | 61531 (41%) | 20360 (14%) |

We claim:

1. A method of forming a circular molecule from a target nucleic acid, comprising:
a) amplifying the target nucleic acid with a first and second bipartite amplification primers comprising a universal circularization sequence and a target-specific sequence to generate double stranded amplicons, wherein only one of the first and second primers comprises a 5′-phosphate group;
b) removing one strand of the double stranded amplicons by nuclease digestion;
c) contacting the remaining strand of the amplicon with a circularization oligonucleotide to generate a hybrid structure wherein the universal circularization sequences in the strand are hybridized to the circularization oligonucleotide so that the ends of the strand are brought into ligatable proximity; wherein the circularization oligonucleotide comprises a ligand for a capture moiety and is attached to a solid support via the capture moiety;
d) ligating the ends of the strand thereby forming a circular molecule.

2. The method of claim 1, wherein the target nucleic acid comprises fragments of a genome selected from cell-free plasma DNA, sonicated DNA and restriction digested DNA.

3. The method of claim 1, wherein the amplification primers further comprise a sequencing primer binding site.

4. The method of claim 1, wherein the ligand-capture moiety pair is selected from biotin-streptavidin, antibody-antigen or oligonucleotide-complementary capture oligonucleotide.

5. A method of determining the sequence of a double-stranded target nucleic acid in a sample comprising:
a) forming a circular molecule according to claim 1, wherein the amplification primers comprise a sequencing primer binding site;
b) contacting the sample with a sequencing primer; and
c) extending the sequencing primer with a nucleic acid polymerase thereby determining the sequence of the target nucleic acid.

6. A kit for determining the sequence of a target nucleic acid comprising:
a) a first and second bipartite amplification primers comprising a universal circularization sequence and a target-binding sequence, wherein only one of the first and second primers comprises a 5′-phosphate group;
b) a circularization oligonucleotide at least partially complementary to the universal circularization sequences in the bipartite primers and further comprising a ligand for a capture moiety.

7. The kit of claim 6 further comprising the capture moiety, a DNA polymerase and a DNA ligase.
